(19) United States
(12) Patent Application Publication
(10) Pub. No.: US 2009/0105754 A1
(43) Pub. Date: Apr. 23, 2009

(54) TENDON REPAIR USING TENSION-SLIDE TECHNIQUE

(76) Inventor: Paul M. Sethi, Cos Cob, CT (US)

Correspondence Address: DICKSTEIN SHAPIRO LLP, 1825 EYE STREET NW, Washington, DC, 20006-5403, US

(21) Appl. No.: 12/251,388

(22) Filed: Oct. 14, 2008

Related U.S. Application Data
(60) Provisional application No. 60/979,703, filed on Oct. 12, 2007.

(51) Int. Cl. A61B 17/04; A61B 19/00

(52) U.S. Cl. 606/228; 128/898; 606/232

(57) ABSTRACT

Tension-slide techniques and reconstruction systems for tendon surgical repairs. The technique improves the biomechanics of the combined fixation and helps overcome surgeons' concerns about rapid return to ADLs. The technique reliably seats the tendon against the distal cortex of the bone socket, maximizing the surface area for bone to tendon healing. The technique takes advantage of cortical fixation, while providing the unique advantage of minimizing gap formation and minimizes surgical dissection by performing the surgery through a single incision technique.

### CROSS-REFERENCE TO RELATED APPLICATIONS

This application claims the benefit of U.S. Provisional Application No. 60/979,703, filed Oct. 12, 2007, the entire disclosure of which is incorporated by reference herein.

### FIELD OF THE INVENTION

The present invention relates to the field of surgery and, more particularly, to tendon repairs for reconstructive surgeries.

### BACKGROUND OF THE INVENTION

Various techniques are known to repair the distal biceps tendon. These techniques include cortical buttons, Bio-Tenodesis screws, bone tunnels, and suture anchors. An optimal technique would be characterized by a limited anterior one-incision, early range of motion due to strength and gapping of the repair, and minimum complications.

The application of cortical button fixation to the biceps and other tendons is an advancement that takes advantage of fixation across strong cortical bone. However, despite the advances, the existing cortical button techniques have inherent problems. In order to seat the tendon flush in the bone socket created for the tendon, a second incision is often made. In addition, extensive dissection and stripping may be needed, while ensuring that the button and suture are readily accessible to tie the tendon down properly. These problems could result in neurologic injury, additional pain, hematoma formation, and delayed function because of dissection.

An alternative method of passing a cortical button involves tying the ends of a whipstitched tendon 10 to a button 125, and then pulling the entire construct through a bicortical hole (FIG. 1). When this technique is performed flawlessly, the tendon 10 will sit in the intramedullary canal of bone 20, with an obligatory minimum 7 mm of suture bridging the tendon to the bone (FIG. 1). Once this construct is cycled, if there is as little as 3 mm of creep or displacement, the tendon button construct 10, 125 is separated by a full 1 cm (FIG. 1), if the technique if flawless. Diastasis between the bone and the tendon could compromise the strength and subsequent healing of the tendon. This technique also hinges on flipping of the button 125 without difficulty and passing a beath pin without injuring the posterior interosseous nerve (which may also prove to be challenging).

The effect of the tendon pistoning in the bone socket also raises concerns, particularly with respect to direct tendon to bone healing. In the specific example of the distal biceps, the tendon is frequently under tension. The button tendon construct may have to be passed with the arm in some degree of flexion, which may obscure visualization, and the tendon may subsequently bunch up against the proximal cortex on the bone. Displacement of as little as one millimeter in this setting creates a potential healing problem in this scenario as well.

In both techniques described above, the surgeon's comfort of allowing early motion is significantly undermined. Early range of motion and early return to ADL's is an ever-present theme in orthopedic surgery; one of the premises behind stronger tendon to bone fixation is to allow early motion. The above mentioned techniques allow early motion in very few cases.

### BRIEF SUMMARY OF THE INVENTION

The present invention provides a tension-slide technique and reconstruction system for tendon repairs. The tension-slide technique is a modified use of a button construct and includes only a transverse anterior incision, allowing tensioning of the button/tendon construct through the anterior incision, as opposed to making a small posterior incision.

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 is a cross sectional image of diastasis between biceps and radial tuberosity after cycling.

FIG. 2 is a cross-section of the radius and distal biceps with a tension-slide repair using multiple sutures and one button, and according to one embodiment of the present invention.

FIGS. 3-10 illustrate various steps during a tension-slide technique using only one suture and one button, and according to another embodiment of the present invention.

FIG. 11 illustrates a perspective view of an exemplary button used with the tension-slide repair of the present invention.

FIG. 12 illustrates a schematic view of a distal biceps repair according to the tension-slide repair of the present invention.

FIG. 13 illustrate a radiograph of a distal biceps repair according to the tension-slide repair of the present invention, and showing the button of FIG. 11 flipped on the tuberosity.

FIGS. 14-23 illustrate various steps during a tension-slide technique and according to another embodiment of the present invention.

FIGS. 24-28 illustrate various views of a button inserter employed for a tendon repair by the tension-slide technique of the present invention.

FIG. 29 illustrates the button inserter of FIGS. 24-28 with a button loaded thereon.

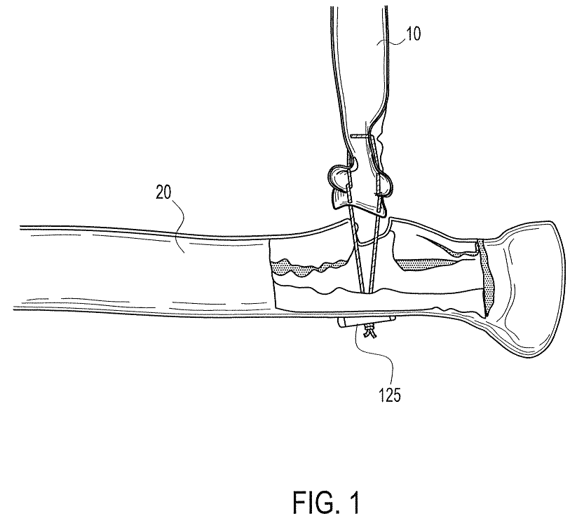

### DETAILED DESCRIPTION OF THE INVENTION

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The present invention provides a tension-slide technique and reconstruction systems for tendon repairs with maximum tendon-to-bone contact. The tension-slide technique is a modified use of a button construct to repair distal biceps tendon ruptures. The tension-slide technique includes only a small, transverse anterior incision, allowing tensioning of the button/tendon construct through the anterior incision, as opposed to making a small posterior incision. The tension-slide technique is an optimal one-incision-only technique with maximal strength and minimal gapping of the repair to allow early range of motion. The tension-slide technique, which allows for a transverse anterior incision and the ability to tension and dock the repair through the anterior incision. There is no need to predetermine the length of suture between the button and the biceps and, thus, the technical concern for the flipping of the button is eliminated. The tension-slide technique eliminates all inherent flaws associated with the conventional biceps repairs.

The technique of the present invention improves the biomechanics of the combined fixation and, thus, helps overcome surgeons' concerns about rapid return to ADLs. The technique reliably seats the tendon against the distal cortex of the bone socket, maximizing the surface area for bone to tendon healing. The technique takes advantage of cortical fixation, while providing the unique advantage of minimizing gap formation, and minimizes surgical dissection by performing the surgery through a single incision. The tension-slide technique restores the anatomical footprint and provides the optimal strength and biomechanical characteristics to allow immediate active range of motion.

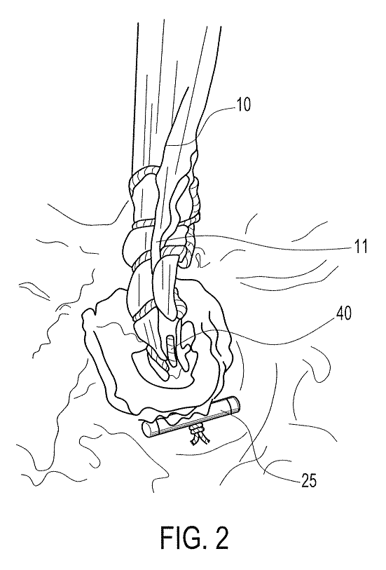

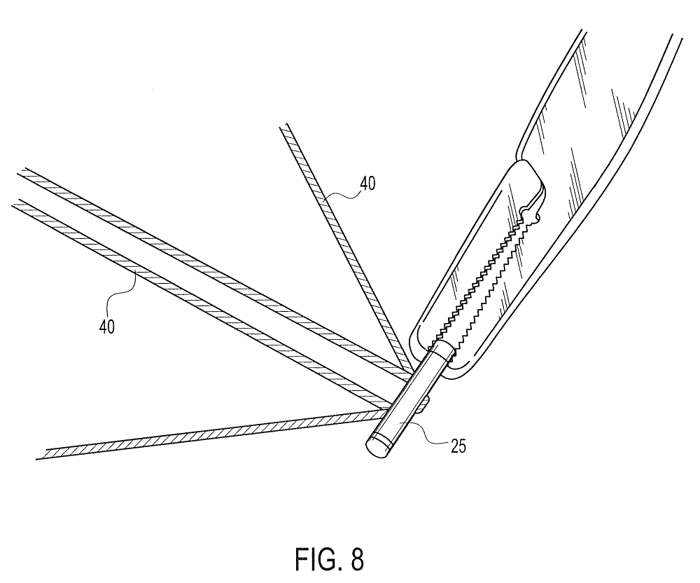

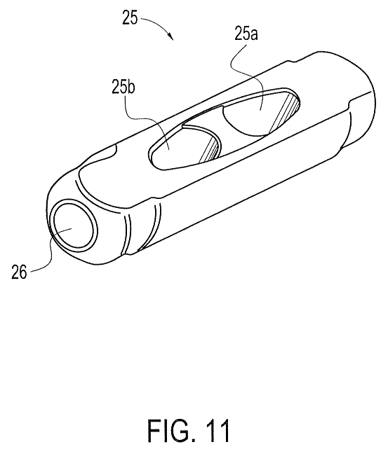

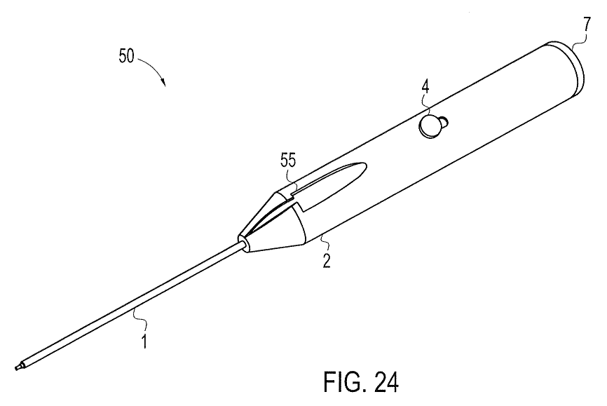

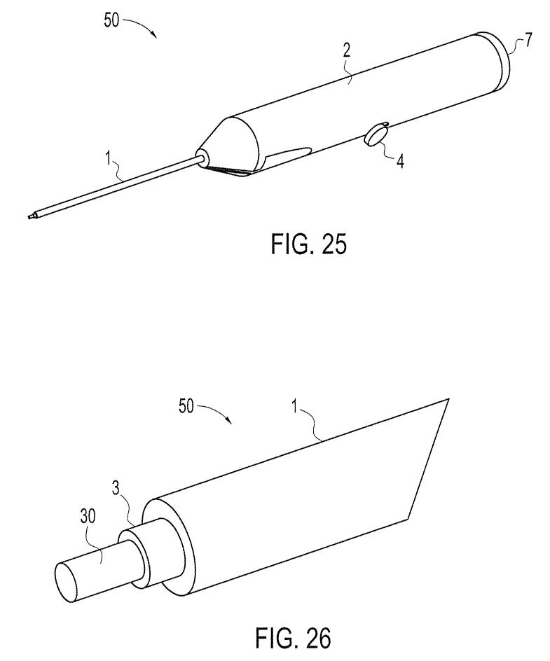

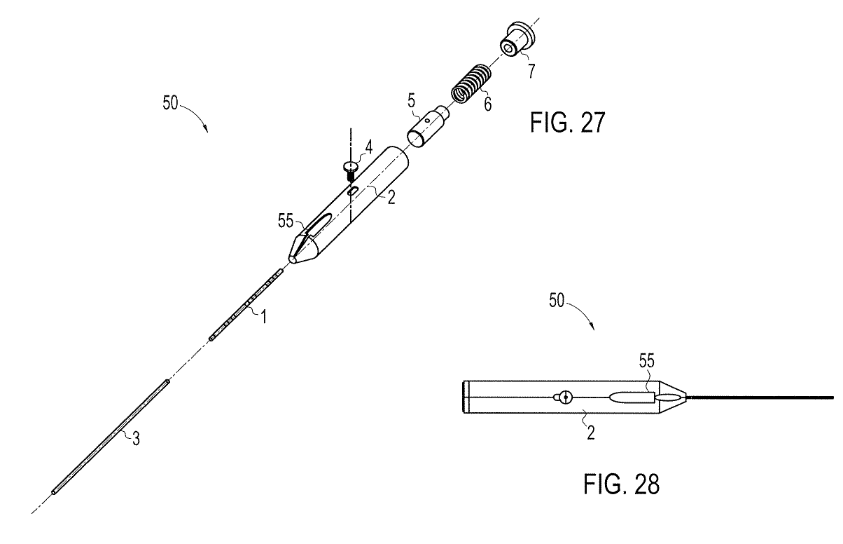

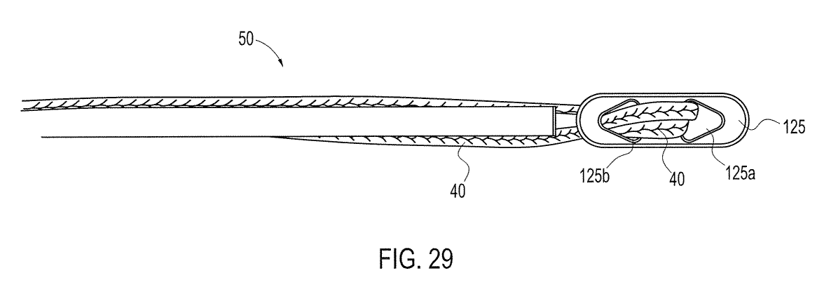

Exemplary methods of a tension-slide technique of the present invention are detailed below with reference to FIGS. 2-23. FIGS. 24-28 illustrate various views of a button inserter 50 which may be employed for a tendon repair using the tension-slide technique of the present invention (to insert button construct 25, 125 (FIGS. 11, 29) attached to tendon 10).

### FIGS. 2-10

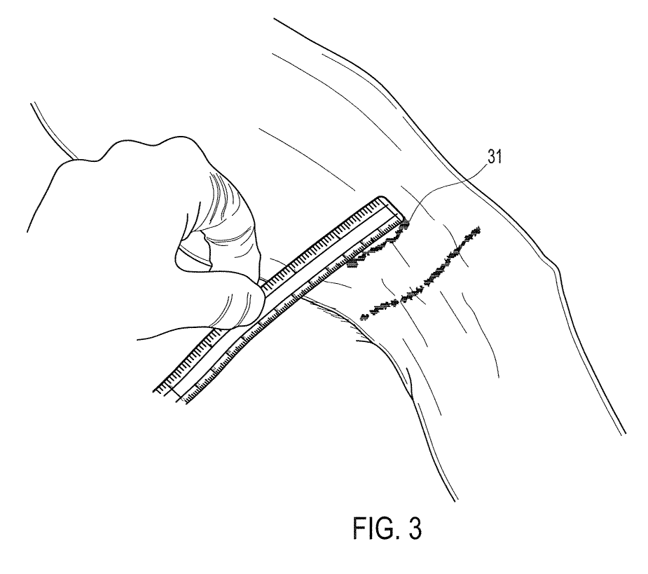

The patient is placed supine on a standard operating room table with a hand extension attachment. A tourniquet is applied, but not regularly inflated. The appropriate extremity is prepped and draped in standard surgical fashion. An anterior incision 31 of about 4 cm is made transversely approximately 4 cm distal to the elbow flexion crease (FIG. 3). The initial incision may be made with a blade. The dissection is carried down through the subcutaneous tissue using dissecting scissors, for example.

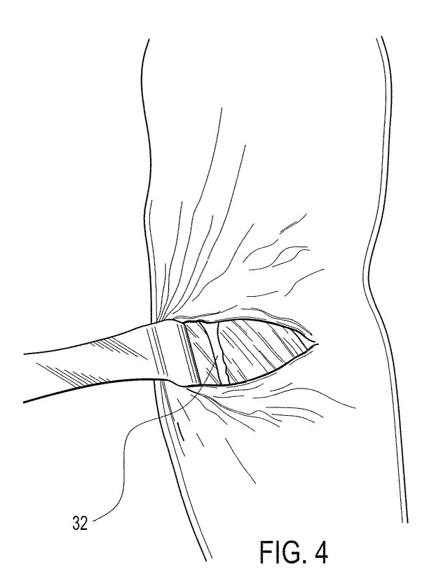

The lateral antebrachial cutaneous nerve 32 is identified and retracted laterally (FIG. 4). The retracted distal end 11 of the biceps tendon 10 is identified after dissection through the antecubital fossa, which may be preceded by identification of a small hematoma or seroma. If desired, the biceps tendon may be “milked” from the wound when not readily identified. Distal end 11 of the biceps tendon 10 may be debrided of excess fibrous and necrotic tissue. A clamp (such as an Alice clamp, for example) may be used to secure the end 11 of the distal biceps tendon 10.

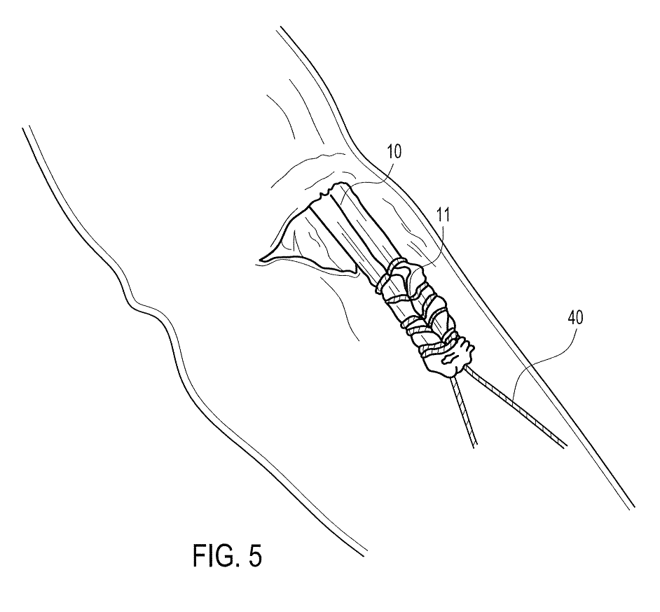

At least one flexible strand 40 (for example, a No. 2 polyester suture such as FiberWire suture) is used to secure the distal end 11 of the biceps tendon 10 (for example, about 2.5 cm) in locking loop fashion (FIG. 5). In a preferred embodiment, the tension-slide technique of the present invention is performed with only one strand of flexible material 40 (for example, only one suture strand) to simplify the technique. The suture may be a single high strength suture such as FiberWire® suture, sold by Arthrex, Inc. of Naples, Fla., and described in U.S. Pat. No. 6,716,234, the disclosure of which is incorporated by reference herein. Preferably, the suture has even tails at the end of the repair and is a #5 FiberWire suture.

In an exemplary embodiment only, the technique may include two strands of flexible material, with four throws heading proximally then returning down with four heading distally. The same or a different technique is used on the second half of the tendon with a second suture (for example, a No. 2 polyester suture). Four strands of the suture will be exiting the tendon distally. Care should be taken to have even suture tails at the end of the repair. Alternatively, the whipstitch may be placed with two loops; this configuration is an effective time-saving operation and is biomechanically advantageous.

The suture 40 is then threaded through a button construct 25, 125 (FIGS. 11 and 29), preferably of titanium alloy (which may additionally have a continuous loop attached to the button). An exemplary embodiment of a button construct 125 is described in U.S. application Ser. No. 11/889,740, filed Aug. 16, 2007, the disclosure of which is herein incorporated by reference. In additional embodiments, the button may be formed PEEK or PLLA, or combination of titanium, PEEK and PLLA. The button may have an oblong configuration and a width that is preferably less than about 1 mm narrower than the width of the hole through which the button is inserted and subsequently passed through. The button is provided with inside eyelets that allows the passage of suture.

Another exemplary embodiment of a button construct 25 employed in the tension-slide technique of the present invention is illustrated in FIG. 11. Button 25 is provided with one or more inside eyelets (for example, a right eyelet or hole 25a, and a left eyelet or hole 25b shown in FIG. 11) that allow the passage of the suture 40. Button 25 is also provided with at least one lateral hole 26 which allows engagement of the button to an inserter instrument (such as applicator 50 of FIGS. 24-28).

Preferably, the button is oriented appropriately with one of the sides toward the biceps tendon 10. In an exemplary embodiment, only one flexible strand 40 (such as a suture strand, for example) is threaded through the button 25. The strand 40 is fed through the right eyelet (or hole) and then back through the left eyelet (or hole). Then, the opposite is performed with the other tail of the same suture, with the strand 40 being fed through the left eyelet (or hole)and then back through the right eyelet (or hole). The end result is to have the strands facing toward the distal biceps tendon 10. Approximately 4-5 cm of space between the button 25 and the end 11 of the biceps tendon 10 should be available to allow for manipulating the button 25 through the radial tuberosity.

According to another exemplary embodiment, four strands of suture are weaved through the button. The first strand is fed through the right hole then back through the left hole. Then, the opposite is performed with the other tail of the same suture (for example, a No. 2 polyester suture) with the strand being fed through the left hole then back through the right hole. Finally, the same is done using the second set of strands from the second suture (for example, a No. 2 polyester suture). The end result is to have the strands facing toward the distal biceps tendon. Approximately 4-5 cm of space between the button and the end of the biceps tendon should be available to allow for manipulating the button through the radial tuberosity. Suture management is important at this point (avoid suture tangles and place a small hemostat on the two sutures coming out of the medial side of button and a separated hemostat on the sutures exiting from the lateral button). A suture loop (for example, a 2-0 FiberLoop) may be threaded through the button. The loop may be used to pull the button through the radial tuberosity.

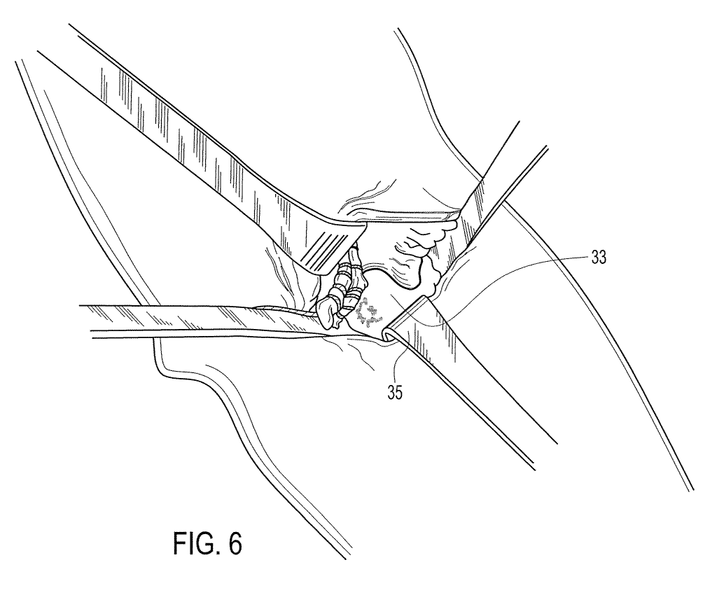

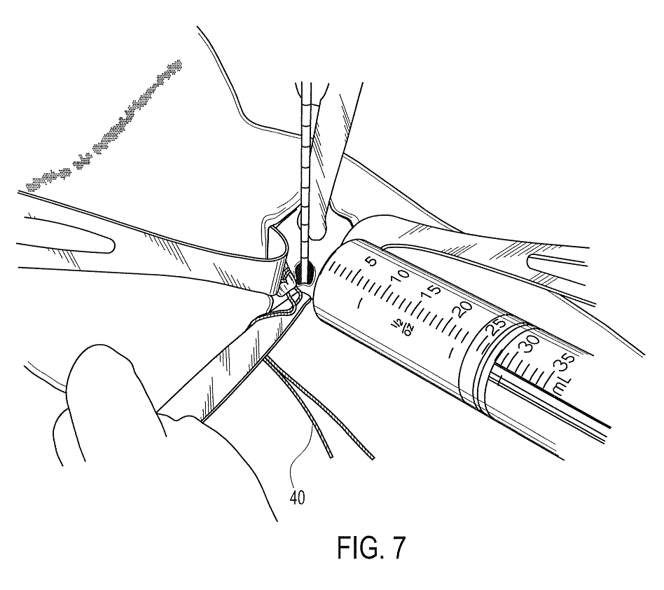

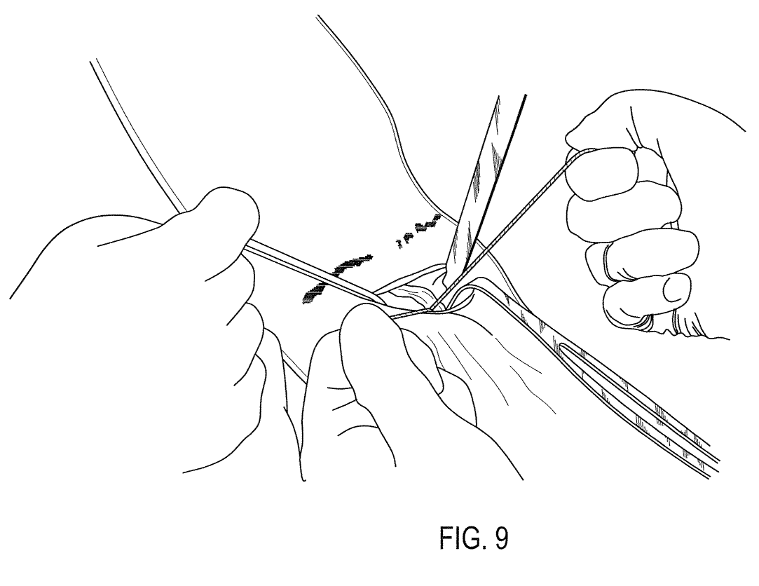

With the elbow in full extension and full supination, the radial tuberosity 33 is exposed and debrided of remaining tissue (FIG. 6). A retractor 35 may be used to minimize nerve and vascular injury. The single strand of suture 40 is then threaded through the button 25. The strand is fed through the right eyelet (or hole) then back through the left eyelet (or hole). Then, the opposite is performed with the other tail of the same suture 40 (for example, a No. 2 polyester suture) with the strand being fed through the left eyelet (or hole) then back through the right eyelet (or hole). The end result is to have the strands facing toward the distal biceps tendon (FIGS. 7 and 9).

A guide pin (for example, a 3.2 mm guide pin) is then drilled through the central aspect of the radial tuberosity from anterior to posterior. Using a cannulated reamer (for example, an 8.0 mm reamer), the anterior cortex and intramedullary canal are then reamed to allow for flush seating of the end of the distal biceps tendon. Keeping the guide pin in place, a cannulated drill bit may then be used to over drill cortices to facilitate easy passage of the button.

Irrigation of the wound to remove bone dust and fragments may be performed at this point. In the exemplary embodiment where a loop is employed (which was threaded through the button), the loop is passed through the eye of the guide pin and tied in place. The guide pin is then fed through the posterior forearm, bringing the button through the radial tuberosity. The radius is maximally supinated, and the pin placed perpendicular to the tunnel to avoid nerve injury. Divergence by more than 30 degree can injure the posterior interosseus nerve. Care should be taken to gently bring the button through the radial tuberosity so as not to pull the button through soft tissue of the extensor mass. Fluoroscopy may be used to visualize this step. Once the button has passed through the radial tuberosity the prolene may be removed from the posterior aspect of the forearm.

Alternatively, a button inserter 50 (or the blunt end of the guide pin) which holds the button 25, 125 may be used to pass the button through the tuberosity, alleviating concerns about nerve injury. An exemplary embodiment of the button inserter 50 of the present invention is illustrated in FIGS. 24-28. Button inserter 50 comprises a shaft 1, a handle 2, an inner rod 3, a bolt 4, a slide 5, a spring 6 and a cap 7. Groove 55 is provided within handle 2 to allow button 25, 125 (FIGS. 11, 29) to rest within the groove. As detailed above, the button is provided with one or more inside eyelets (for example, a right eyelet or hole 25a, 125a and a left eyelet or hole 25b, 125b shown in FIGS. 11, 29) that allow the passage of the suture 40. Details of the button inserter 50 are set forth in U.S. patent application Ser. No. 12/167,922, filed Jul. 3, 2008 (Attorney Docket No. A8130.0546/P546), entitled “Applicator for Suture/Button Construct,” the entire disclosure of which is incorporated by reference herein.

The button 25, 125 is released from the applicator 50 and a tactile release of the button may be sensed. Fluoroscopy may be used to visualize the button at this step. The button 25, 125 is tested at this point by pulling back on the suture limbs.

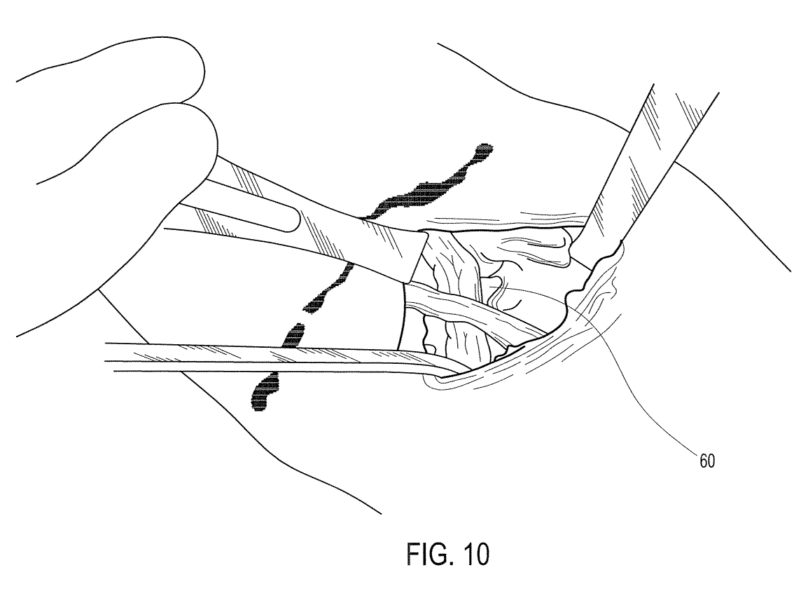

As shown in FIG. 9, one limb of each suture 40 is then grasped in each hand and slowly tensioned. As this step is performed, the biceps will dock itself in the prepared bone socket. The arm may be flexed about 20-30 degrees so that the tendon slides into the bony socket (FIG. 9). In a low demand patient, a free needle may then be used to pass one end of the sutures (for example, the No. 2 polyester sutures) through the biceps tendon closest to the biceps tuberosity and tied. In higher demand patients, a fixation device 60 (for example, an interference screw such as a 7 by 10 mm interference screw) is then inserted on the radial side of the tendon 10, and the suture limbs 40 are additionally tied over the fixation device 60. The fixation device 60 is left flush with the anterior cortex (FIG. 10). The elbow should be taken through a full range of motion to ensure that the tendon is secure.

Tears that are more than about 4 weeks old, or tears that have inelastic tendons, require additional caution when using the technique of the present invention. To avoid suture breakage, it is important to pull the suture in line with the tendon (like aligning a cannula with arthroscopic knots) and to avoid the suture dragging over the posterior cortex of the radius. In situations where the construct does not slide easily, a “rescue suture” may be applied. After the above-mentioned preparation, a single heavy suture is placed through the tendon. The rescue suture is then passed through the hole in the radial tuberosity with a needle, for example, and pulled out percutaneously. The rescue suture may then be tensioned (in line with the biceps) to help guide the reduction. Once the construct is affixed, the rescue suture may be pulled out of the forearm and discarded. The wound is closed and a soft dressing may be applied. The patient may be placed in a sling, for comfort.

In the exemplary embodiment where two or more flexible strands are used, the tension-slide technique is also used on the anterior aspect of the forearm through the transverse incision. With one hand gripping the medial two sutures (for example, No. 2 polyester sutures) and the other gripping the lateral two sutures (for example, No. 2 polyester sutures), the strands are toggled with equal tension to pull the gap out of the suture between the distal biceps tendon and the button. A tonsil snap may be used to assist in directing the most distal aspect of the distal biceps tendon into the hole drilled through the radial tuberosity. The arm may be flexed to about 20-30 degrees so that the tendon slides into the bony socket. The tendon appears taught at the end of the technique. The gap is eliminated from the repair.

A free needle may then be used to pass one end of one of the sutures (for example, the No. 2 polyester sutures) through the biceps tendon closest to the biceps tuberosity. This first suture should then be tied with a plurality of throws (for example, at least five throws), while tension is held on the second suture. The same or different technique may be performed with the second suture: using a free needle, pass one end of the suture through the distal biceps tendon, then tie the suture using the plurality of throws. Leaving a tail, the end of the suture is cut with a scalpel, for example. A fixation device (for example, an interference screw) is then inserted on the radial side of the tendon depending on the size of the tendon and quality of the bone. This screw is left flush with the proximal (anterior) cortex. The elbow should be taken through a full range of motion to ensure that the tendon is secure.

The suture limbs are tied down, after passing one limb through the tendon, prior to placing the fixation device. When a longer screw is employed, it may not be necessary to tie the sutures as the interference screw may obviate the need for suture tying.

The area may be irrigated with normal saline. The tourniquet, if applied, is released, and any small venous or arterial bleeders are cauterized. The subcutaneous tissue is closed with suture and then the skin is closed using a stitch (for example, a subcuticular stitch). Sterile strips may be placed. The area may be wrapped with dry sterile dressing and a soft dressing. The patient may be placed in a sling, for comfort.

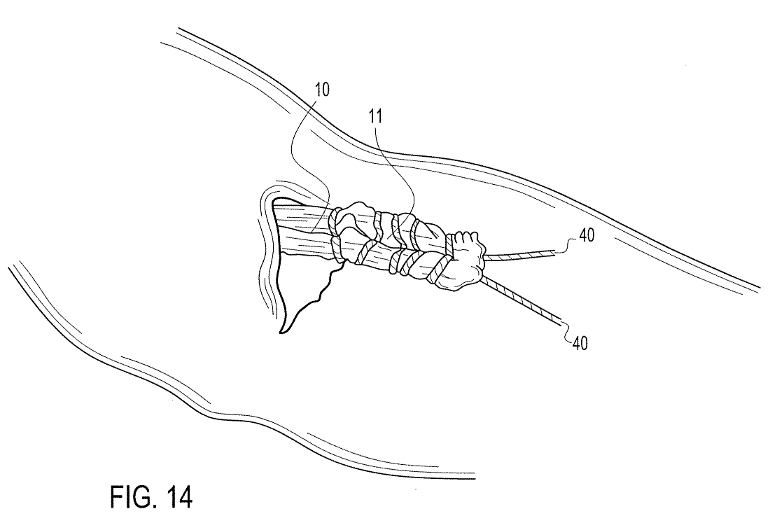

### FIGS. 14-23

FIGS. 14-23 illustrate various steps of another method of a tension-slide technique, more clearly illustrating the use of a fixation device such as a tenodesis screw, to allow stronger repairs and better anatomical positioning of the tendon. As in the previously-described embodiment, the tendon end is first identified and retracted, and then debrided, measured and trimmed to about 8 mm thickness, if necessary. If the tendon edge appears degenerative, about 1 cm of the tendon may be resected. A clamp may be used to secure the end 11 of the tendon 10 during whipstitching.

The biceps tendon 10 is whipstitch using a suture strand (for example, a FiberLoop). About 2.5 cm of the tendon end may be whipstitch. The final pass through the distal end of the tendon should be passed proximal to second to last stitch, thus creating a locked configuration. The strand (FiberLoop) is cut to create two independent suture limbs 40 (FIG. 14).

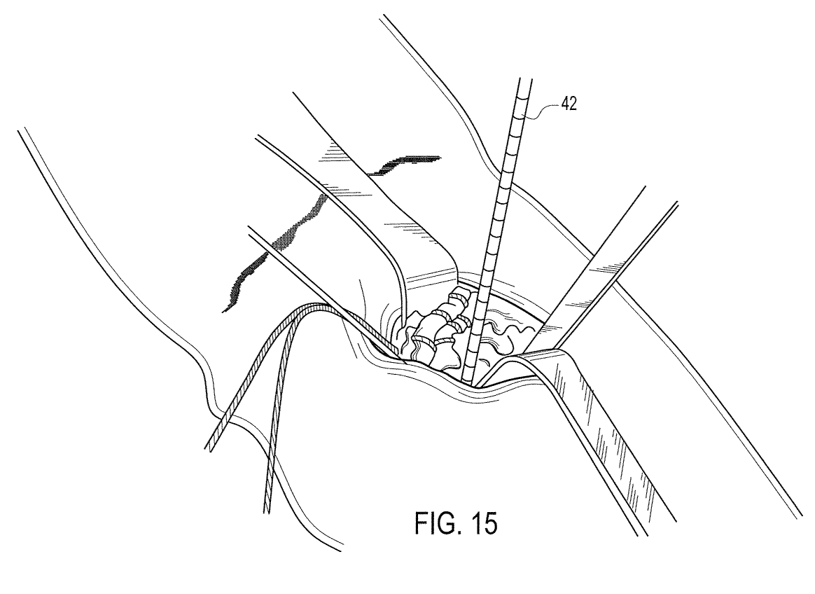

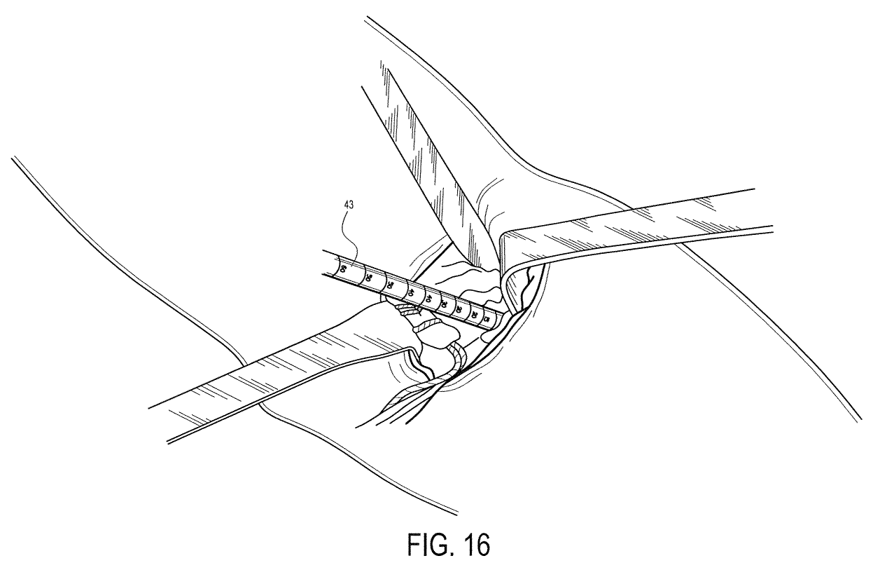

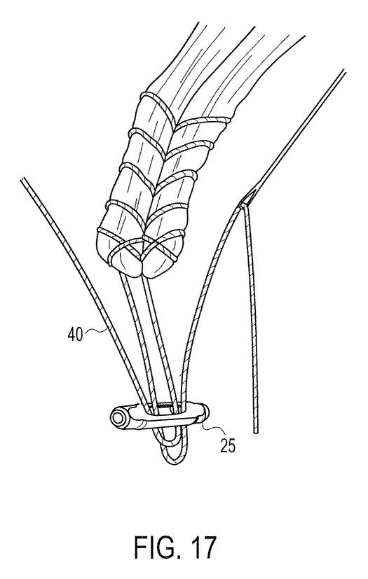

A drill 42 (for example, a 3 mm drill) is drilled through both cortices, and then the drill is left in place (FIG. 15). A reamer (for example, a 7.5 or 8 mm cannulated reamer) 43 is used to ream over the pin through the first cortex only, leaving the pin in (FIG. 16). As shown in FIG. 17, button 25 is threaded with the two suture limbs 40 (from the cut FiberLoop). The first limb (strand) is fed through the left hole of the button 25 and back through the right hole. The opposite is performed with the other tail of the strand (#2 FiberWire), with the strand being fed through the right hole and back through the left hole. The end result is to have the strands facing toward the distal biceps tendon. About 4-5 cm of space between the button and the end of the biceps tendon 10 should be available, to allow for the manipulation of the button through the radial tuberosity.

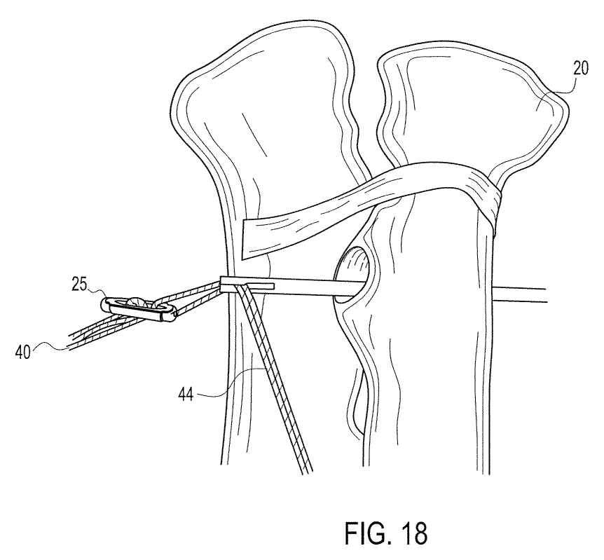

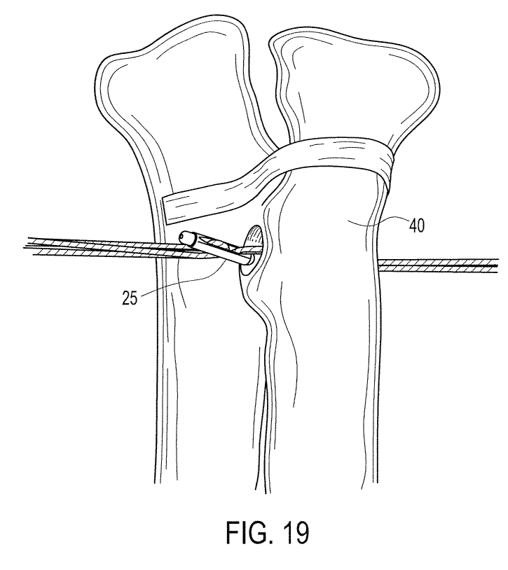

A pull suture 44 (for example, a #2 FiberWire) is loaded through the button 25 (only in one hole of the button) and pin eyelet (FIG. 18) of the drill pin. The pull suture is used to pull the button 25 through the reamed socket and the distal cortex. Care must be taken not to draw the button through the dorsal soft tissue. This may be avoided by holding “back tension” against the sutures and tendon exiting the reamed socket. The button 25 is then drawn with the pull suture 44 and through the radius 20, subsequent to which the pull suture 44 is removed (FIG. 19). Fluoroscopy may be used to visualize the button at the end of this step and to verify close apposition to bone. The button may be tested by pulling back on the suture limbs and tendon exiting the reamed socket.

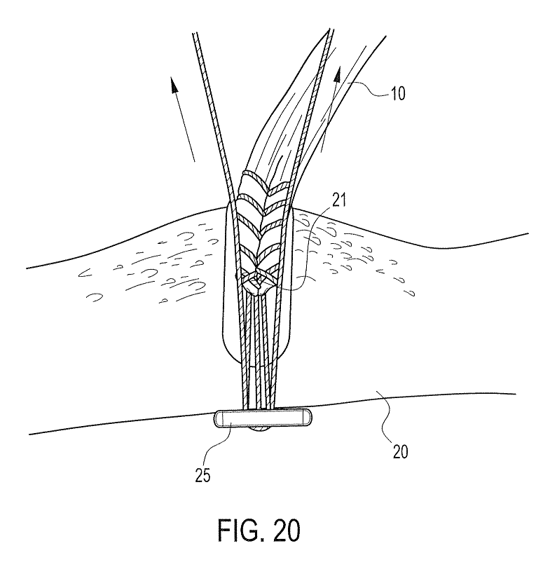

The suture limbs 40 are tensioned to draw the biceps tendon 10 into prepared socket 21 (FIG. 20). As each of the suture limbs is slowly tensioned (grasped in each hand), the biceps tendon will dock itself into the prepared bone socket. A hemostat may be used to assist in directing the most distal aspect of the tendon into the hole drilled through the radial tuberosity. Flexing the arm for about 20-30 degrees may help sliding the tendon into the bony socket. The tendon should appear taut at the end of this step. Pull suture 44 is then removed (FIG. 19).

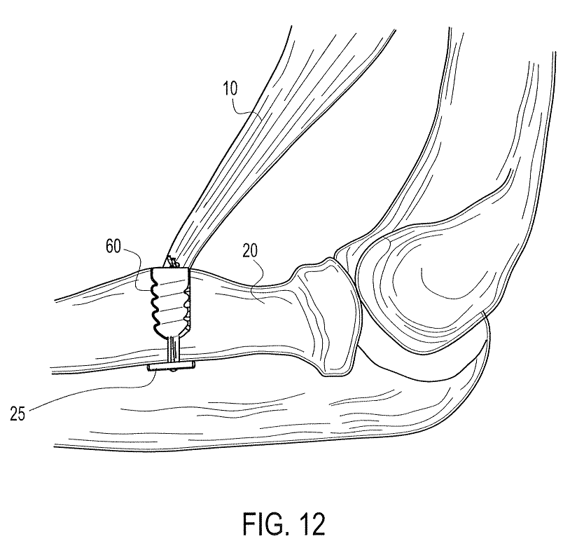

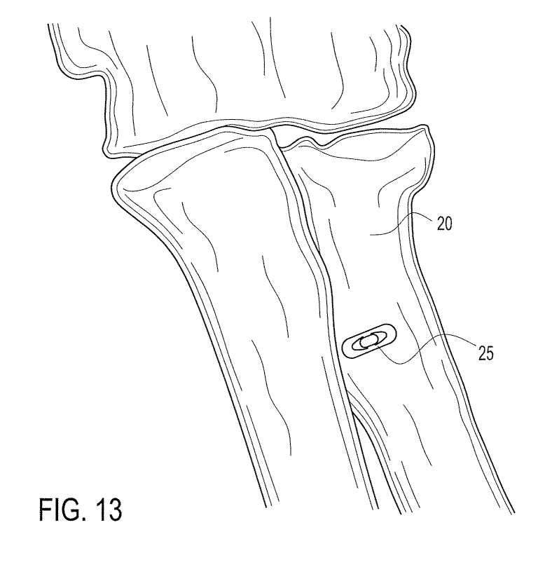

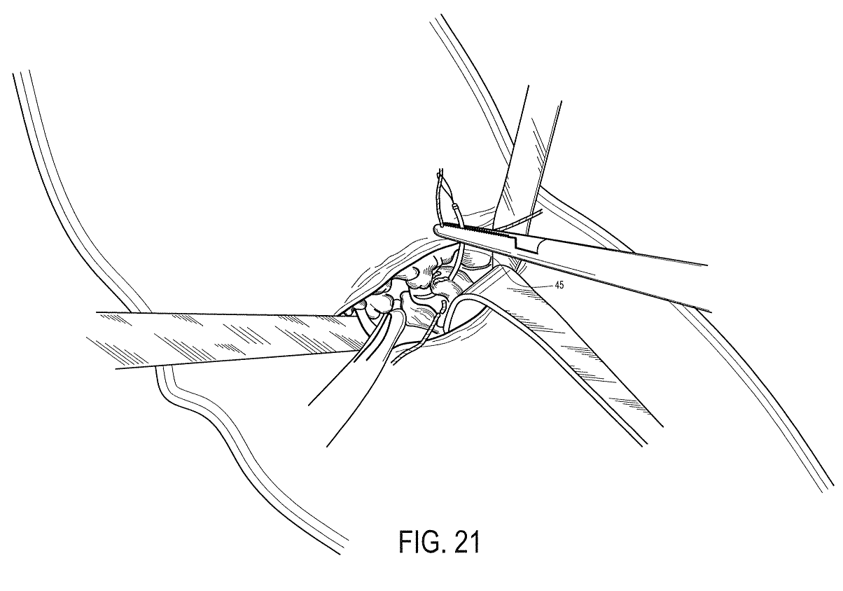

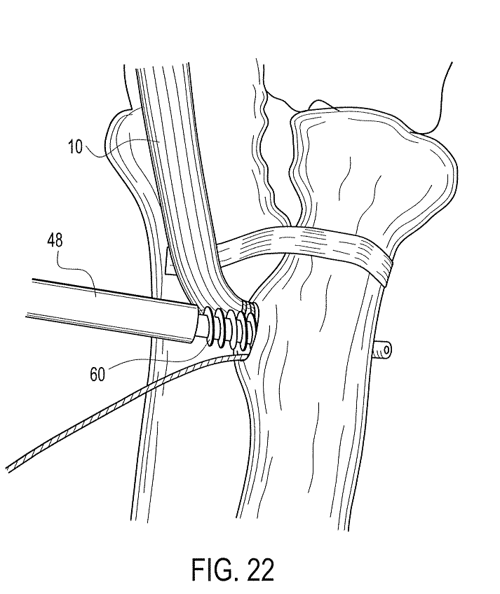

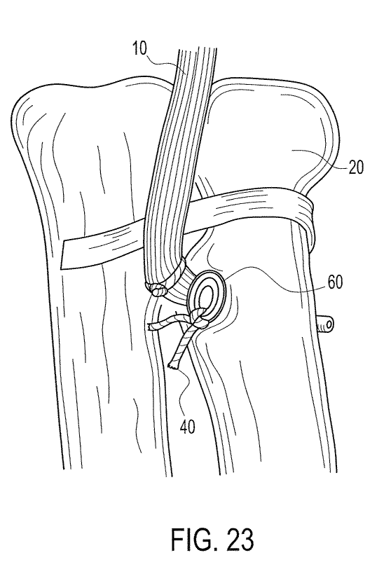

A free needle 45 may be used to pass one suture limb 40 through the tendon 10, while the second suture limb 40 is tied (FIG. 21). One suture limb is run through the driver 48 (for example, a tenodesis screw/driver) and passed through it, and then inserted on the radial side of the tuberosity (thus pushing tendon 10 to the ulnar side). The fixation device 60 (for example, a screw) is inserted while pushing the tendon 10 to the ulnar side of the radius 20 (FIG. 22). The suture limbs 40 are tied together over the top of the fixation device (screw) and then cut to complete the repair (FIG. 23). Various views of the final repair according to the tension-slide of the present invention are illustrated in more details in FIGS. 12 and 13.

The tension-slide technique is a novel procedure to repair distal biceps tendon ruptures. The advantages of the technique include: a small one-incision anterior approach, the ability to tension the repair from the anterior incision, and the utilization of the strength of the button construct.

The goal of any tendon repair is to restore the anatomy and function of the tendon. The biceps is not a cylindrical tendon that inserts onto the radial tuberosity; it is a ribbon-like insertion that is on the ulnar side of the tuberosity. As such, the placement of a fixation device (such as an interference screw) in the tuberosity (on the radial side of the tendon) is important in helping to restore the pre-injury anatomy. The screw allows for proximal cortical fixation and the button completes bicortical fixation of the tendon, the long time goal for fracture fixation. Further, after the time zero strength, interference screw fixation has been histologically linked with direct tendon-to-bone healing.

In the tension-slide technique of the present invention, gap formation is minimized and almost eliminated as the surgeon is able to tension the distal biceps tendon/button complex through the anterior incision, thus setting the button flush against the posterior aspect of the radial tuberosity. By minimizing the gap formation and continuing to have the highest load to failure, the tension-slide technique is an optimum procedure to repair a distal biceps tendon rupture. Laboratory investigations show superior performance of this construct with respect to gap formation and load to failure.

By employing the button inserter and passer instrument shown in FIGS. 24-28, the tension-slide technique of the present invention eliminates the need to pass a beath pin or needle through the forearm, further minimizing the risk of nerve injury. In contrast, the conventional two-incision technique is associated with proximal radio-ulnar synostosis. This may be caused by injury to the interosseous membrane, in combination with bone debris and hematoma lying between radius and ulna and stimulation of the ulnar periosteum by the dorsal exposure. In the dual-incision technique, supination strength may be compromised as a result of non-anatomic position of the tendon repair.

The repair of the present invention offers the highest strength and no gap formation, which is ideal to allow patients to flex/extend and pronate/supinate their elbow immediately after surgery and limit the rate of re-rupture.

Biomechanical data on the tension-slide technique of the present invention indicate increased performance. Loads to failure were between 328.76 for a single suture and button, and 432.23 with the addition of an interference screw; all these were associated with very low standard deviations. The combination of the button and screw offers excellent strength with only one suture and minimal gap formation. A single suture simplifies the procedure, leaves less foreign body in the native tendon and (combined with the screw) eliminates gap formation with cyclical loading.

Displacement after cyclical loading has important consequences in the setting of early postoperative range of motion and on healing. Standard techniques with cortical button fixation (i.e., not using the tension-slide technique of the present invention) have reported 2.59 mm after only 1000 cycles (as detailed by Sprang J T, Weinhold P S, Karas S G in A biomechanical comparison of EndoButton versus suture anchor repair ofdistal biceps tendon injuries (J Shoulder Elbow Surg. 2006; 15:509-514)). Mazzocca et al. reported that the endobutton had the second highest displacement (3.42 mm) as compared with the bone tunnel (3.55 mm), suture anchor (2.33 mm), and interference screw (2.14 mm) (Mazzocca A D, Burton K J, Romeo A A, et al. Biomechanical evaluation of 4 techniques ofdistal biceps brachii tendon repair, Am. J Sports Med. 2007; 35:252). Close to 30% of suture anchor repairs in this series failed during cyclical loading. A recent study comparing two different suture types fixed to a bone tunnel yield 6.8 to 6.9 mm of tendon displacement before failure, noting failure of bone tunnel fixed with fiberwire during cyclical loading (Bisson L J, de Perio J G, Weber A E, et al. Is it safe to perform aggressive rehabilitation after distal biceps tendon repair using the modified 2-incision approach? Am. J Sports Med. 2007; 35:2045-2050). All these methods suggest that pistoning of the tendon occurs during early motion. This macromotion delays and inhibits direct tendon healing.

In the tension-slide technique of the present invention, gap formation between the biceps tendon and radial tuberosity is minimized. Gapping was measured between 1.25 and 1.63 mm after 3600 cycles; this result is superior to all other tested and reported studies that evaluated gap formation. The ability to tension the distal biceps tendon/button complex through the anterior incision and dock the tendon flush against the posterior aspect of the radial tuberosity is unique to this procedure and plays an important role in minimizing gap formation. By severely minimizing the gap formation and maintaining the highest load to failure, the tension-slide technique of the present invention is the optimum procedure to repair a distal biceps tendon rupture. None of the specimens tested failed during cyclical loading offering advantage over both suture anchor and bone tunnel fixation. This result suggests that the tension-slide technique of the present invention is very durable under cyclical conditions, designed to mimic early postoperative range of motion.

The addition of the interference screw (added to the ultimate tensile load) reduced the gap formation and improved the stiffness of the construct. An immediate active range of motion is provided by placing only a soft dressing at the time of surgery to allow the patient directed range of motion. The patient was restricted from lifting anything more than about 5 pounds before the first postoperative visit, typically about 10 days after surgery. No failures were reported. The tension-slide technique is useful for the acute tear, and a wider exposure and alternate techniques may be additionally employed in tears more than four weeks old.

The tension-slide technique of the present invention repairs distal biceps tendon ruptures. The advantages of the technique include a small one incision anterior approach, the ability to tension the repair from the anterior incision, and the utilization of the strength of cortical button fixation. These is no need to predetermine the length of suture between the button and the biceps, and there is no concern about the button flipping. These advantages, combined with the superior biomechanical performance of the repair, confer an improved technique on acute distal biceps repairs. All patients were started on immediate activity of daily livings and unrestricted range of motion with no brace or sling after surgery, without any clinical failures.

The tension-slide technique of the present invention restores the biceps anatomy to the ulnar side of the radial tuberosity, takes advantage of superior biomechanics, relies on bicortical fixation, and allows immediate postoperative range of motion.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed as new and desired to be protected by Letters Patent of the United States is:

1. A method of conducting arthroscopic surgery, comprising the steps of:
forming a bone socket in a bone adjacent a tendon or ligament;
conducting a first incision in the vicinity of the bone socket, without conducting a second incision; and
securing the tendon or ligament to a button construct having at least one eyelet.

2. The method of claim 1, wherein the first incision is a transverse anterior incision.

3. The method of claim 1, wherein the tendon or ligament is a biceps tendon.

4. The method of claim 1, further comprising the steps of:
attaching a distal end of the tendon to a suture;
attaching the suture and the distal end of the tendon to the button construct, to form a tendon/button construct; and
subsequently, positioning the tendon/button construct within the bone socket.

5. The method of claim 4, further comprising the steps of:
pulling the tendon/button construct through the bone socket;
positioning the tendon/button construct within the bone socket; and
securing the button to the bone cortex abutting the bone.

6. The method of claim 1, wherein the button construct further comprises a suture loop attached to the button.

7. The method of claim 6, wherein the suture loop is formed of a suture material comprising ultrahigh molecular weight polyethylene.

8. The method of claim 1, wherein the button construct has an oblong or round configuration.

9. The method of claim 1, wherein the button construct has a length of about 10 to about 20 mm.

10. The method of claim 1, wherein the button construct has a width that is less than about 1 mm narrower than a width of the bone socket.

11. A method of tissue reconstruction, comprising the steps of:
forming a socket or tunnel within a bone;
conducting a transverse anterior incision in the vicinity of the socket or tunnel, without conducting an additional incision;
providing the tissue in the vicinity of the socket or tunnel;
securing a distal end of the tissue to a suture loop/button construct comprising a button having at least one eyelet, and a continuous suture loop attached to the eyelet;
securing the button of the suture loop/button construct to a bone cortex abutting the socket or tunnel; and
securing the tissue within the socket or tunnel.

12. The method of claim 11, wherein the tissue is biological or non-biological tissue.

13. The method of claim 11, wherein the tissue is at least one of ligament, tendon, bone and cartilage.
